(19) United States
(12) Patent Application Publication
(10) Pub. No.: US 2026/0345151 A1
(43) Pub. Date: Nov. 5, 2026

(54) TISSUE SUTURING DEVICE AND OPERATING METHOD THEREOF AND OPERATING ASSEMBLY

(71) Applicant: Cilag GmbH International, Zug (CH)

(72) Inventors: Jie ZHANG, Shanghai (CN); Sijin HE, Shanghai (CN); Wencan LI, Shanghai (CN)

(21) Appl. No.: 19/486,050

(22) PCT Filed: May 24, 2024
(86) PCT No.: PCT/EP2024/064333

§ 371 Date: Nov. 20, 2025

(30) Foreign Application Priority Data
May 24, 2023 (CN) 202310594939.4

(51) Int. Cl. A61B 17/04; A61B 17/00; A61B 17/06; A61B 17/062; A61B 90/00

(52) U.S. Cl. CPC A61B 17/0469; A61B 17/0491; A61B 17/0493; A61B 17/06061; A61B 17/0625; A61B 90/03; A61B 90/08; A61B 2017/00367; A61B 2017/00477; A61B 2017/0472; A61B 2090/035; A61B 2090/0801; A61B 2090/0807

(57) ABSTRACT

The present invention relates to the technical field of surgical instruments. Disclosed are a tissue suturing device and an operating method thereof and an operating assembly. The operating assembly includes an initial position, and a wing driving phase, a transition phase, and a needle driving phase which are sequentially implemented. In the initial position, a needle operating member is in a first locking state in which axial movement of the needle operating member is prevented; in the wing driving phase, a wing operating member moves axially from a third position to a fourth position; in the transition phase, the wing operating member drives the needle operating member to rotate by a predetermined angle, such that the wing operating member and a tube body are in a third locking state in which rotation and axial movement of the wing operating member are prevented, so as to be maintained in the fourth position, and the needle operating member and the tube body are unlocked to be axially movable only; and in the needle driving phase, the needle operating member moves axially from a first position to a second position. The operating assembly can sequentially operate stabilizing wings and suturing needles, and avoid accidental extension of the suturing needles, thereby significantly improving the safety of using the tissue suturing device.

### TECHNICAL FIELD

The present invention relates to the technical field of surgical instruments, and specifically relates to an operating assembly for a tissue suturing device, a tissue suturing device, and an operating method of a tissue suturing device.

### BACKGROUND OF THE INVENTION

During an operation, surgeons usually need to use tissue closure instruments to suture a patient's wound to be sutured, such as an incision of the abdominal cavity.

Existing tissue closure instruments typically include a cannula, stabilizing wings, and a puncture needle. The stabilizing wings are arranged at a distal end of the cannula, and the puncture needle is arranged within the cannula and can extend out of the cannula. When in use, an operator inserts the cannula into the wound to be sutured, and then operates the tissue closure instrument to unfold the stabilizing wings on the inside surface of the tissue of the wound to be sutured, thereby stabilizing the cannula. Then, the puncture needle is operated to extend out of the cannula and pass through the tissue to reach the stabilizing wings, thereby completing the arrangement of the suture. During actual suturing, a skilled surgeon can achieve correct puncture of the puncture needle by virtue of experience. For many surgeons, however, due to various factors such as inexperience or accidental contact, the suturing needle will usually be extended out of the cannula before the stabilizing wings are unfolded, which may cause unnecessary injury to patients or the surgeons themselves.

### BRIEF SUMMARY

In view of the above problems existing in the prior art, a first aspect of the present invention provides an operating assembly for a tissue suturing device. The operating assembly can sequentially operate the stabilizing wings and the suturing needle, and avoid accidental extension of the suturing needle, thereby significantly improving the safety of using the tissue suturing device.

In order to achieve the objective described above, the present invention provides an operating assembly for a tissue suturing device. The operating assembly comprises a tube body, a wing operating member, and a needle operating member, wherein the tube body comprises a proximal end, a distal end, and an axial channel extending between the proximal end and the distal end; the wing operating member is axially and movably installed at the axial channel and is rotatable; and the needle operating member is arranged in the wing operating member and is rotatable along with the wing operating member and is axially movable relative to the wing operating member. The operating assembly comprises an initial position, and a wing driving phase, a transition phase, and a needle driving phase which are sequentially implemented, wherein in the initial position, the needle operating member and the tube body are in a first locking state, in which the needle operating member is prevented from moving axially towards the distal end so as to maintain the needle operating member in a first position, the first position being used to enable suturing needles to be in a retracted state, and the wing operating member and the tube body are in a second locking state, in which the wing operating member is prevented from rotating and is allowed to move axially towards the distal end so as to maintain the wing operating member in a third position, the third position being used to enable stabilizing wings to be in a folded state; in the wing driving phase, the wing operating member moves axially towards the distal end from the third position to a fourth position so as to be unlocked from the second locking state, the fourth position being used to enable the stabilizing wings to be in an unfolded state; in the transition phase, the wing operating member drives the needle operating member to rotate by a predetermined angle, such that the wing operating member and the tube body are in a third locking state in which rotation and axial movement of the wing operating member are prevented, so as to be maintained in the fourth position, and the needle operating member and the tube body are unlocked to be axially movable only towards the distal end; and in the needle driving phase, the needle operating member moves axially towards the distal end from the first position to a second position, the second position being used to enable the suturing needles to be in an extended state.

In this technical solution, the operating assembly comprises the initial position, and the wing driving phase, transition phase, and needle driving phase which are sequentially implemented, and the wing operating member is configured to drive a wing driving member to move axially only in the wing driving phase, while the needle operating member is configured to drive a needle mounting member to move axially only in the needle driving phase. As a result, during actual use, in the wing driving phase, only the wing operating member can be driven to move axially, and the needle operating member cannot be driven to move axially. In this case, even if the needle operating member is incorrectly operated or accidentally touched, the needle operating member cannot drive the needle mounting member to move axially, and the suturing needles mounted on the needle mounting member thus cannot extend out of the tube body. In this way, accidental extension of the suturing needles can be completely avoided, thereby significantly improving the safety of using the tissue suturing device. The transition path can allow the needle operating member to move axially only. In this way, in the needle driving phase, the needle operating member can move axially from the first position to the second position, such that the suturing needles extend out of the tube body.

In some embodiments, in at least two locking states among the first locking state, the second locking state, and the third locking state, locking is implemented based on a same locking part of the tube body.

In some embodiments, in the first locking state, the second locking state, and the third locking state, locking is implemented based on the same locking part.

In some embodiments, the locking part is a locking block extending inwards radially from an inside surface of an axial sidewall of the tube body.

In some embodiments, in the fourth position, an end surface of the proximal end is in contact with a distal end surface of a grip part of the wing operating member to stop axial movement of the wing operating member.

In some embodiments, a locking protrusion is formed on one of the wing operating member and the tube body, and a locking recess is formed on the other of the wing operating member and the tube body, and when the wing operating member rotates to drive the locking protrusion to be pressed into or out of the locking recess, a tactile feedback of being in or out of the third locking state is provided.

In some embodiments, an axially extending guide slot is formed on one of the needle operating member and the wing operating member, and a guide block is formed on the other of the needle operating member and the wing operating member, and the guide block fits into the guide slot, such that the needle operating member is rotatable along with the wing operating member and axially movable relative to the wing operating member.

In some embodiments, in the second position, a proximal slot wall of the guide slot is in contact with the guide block to stop the needle operating member from moving axially towards the distal end.

In some embodiments, a first proximal axial slot and a first distal axial slot communicating with each other are formed on an outer peripheral surface of the wing operating member; the circumferential dimension of the first proximal axial slot is greater than the circumferential dimension of the first distal axial slot, so that a first stopper platform is formed therebetween; in the initial position, the locking part of the tube body fits into the first distal axial slot to prevent rotation of the wing operating member, and the locking part is axially aligned with the first proximal axial slot to enable the wing operating member to move axially towards the distal end, such that the wing operating member and the tube body are in the second locking state; in the wing driving phase, the wing operating member moves axially towards the distal end, such that the locking part enters the first proximal axial slot from the first distal axial slot; and in the transition phase, after the wing operating member rotates by a predetermined angle, the locking part and the first stopper platform are locked, such that the wing operating member and the tube body are in the third locking state.

In some embodiments, a locking recess is formed on one of a surface of the first stopper platform and a distal end surface of the locking part, and a locking protrusion is formed on the other of the surface of the first stopper platform and the distal end surface of the locking part, wherein when the wing operating member rotates to drive the locking protrusion to be pressed into or out of the locking recess, the tactile feedback of being in or out of the third locking state is provided.

In some embodiments, a second proximal axial slot and a second distal axial slot communicating with each other are formed on an outer peripheral surface of the needle operating member; the circumferential dimension of the second proximal axial slot is less than the circumferential dimension of the second distal axial slot, such that a second stopper platform is formed therebetween; in the initial position, a locking part of the tube body fits into the second distal axial slot and is engaged with the second stopper platform, such that the needle operating member and the tube body are in the first locking state; in the transition phase, the wing operating member drives the needle operating member to rotate by a predetermined angle, such that the locking part is separated from the second stopper platform and axially aligned with the second proximal axial slot to unlock the needle operating member and the tube body; and in the needle driving phase, the locking part and the second proximal axial slot are axially movable relative to each other.

In some embodiments, the tube body comprises two half tubes capable of being joined together, wherein each of the half tubes is capable of being in the second locking state and the third locking state together with the wing operating member and being in the first locking state together with the needle operating member.

In a second aspect, the present invention provides a tissue suturing device. The tissue suturing device comprises the operating assembly according to any of the foregoing described in the first aspect above, a wing driving member, stabilizing wings, a needle mounting member, and suturing needles, wherein the wing driving member is axially and movably arranged in the axial channel, the stabilizing wings are hinged to the distal end of the tube body and hinged to the wing driving member, the needle mounting member is axially and movably arranged in the axial channel, and the suturing needles are arranged on the needle mounting member and located in the tube body; wherein the wing operating member is rotatably connected to the wing driving member, and the needle operating member is rotatably connected to the needle mounting member; wherein the wing operating member is capable of driving, by means of the wing driving member, the stabilizing wings to be unfolded and folded, and the needle operating member is capable of driving, by means of the needle mounting member, the suturing needles to extend out of the tube body to a puncture position and to retract back into the tube body.

In this way, as described in the first aspect above, by means of the operating assembly, the tissue suturing device can completely avoid accidental extension of the suturing needle, thereby significantly improving the safety of using the tissue suturing device.

In some embodiments, the wing driving member is formed with an axially extending hollow channel, wherein the needle mounting member is arranged in the hollow channel in an axially movable manner, and the needle operating member is connected to the needle mounting member and is capable of extending axially into the hollow channel.

In some embodiments, an elastic member is arranged between the wing driving member and the tube body, wherein when the wing operating member drives the wing driving member to move axially from the third position to the fourth position, the elastic member stores energy; and when the wing operating member and the tube body are unlocked from the third locking state, the elastic member releases energy to drive, by means of the wing driving member, the wing operating member to return from the fourth position to the third position.

In some embodiments, when the elastic member fails or malfunctions, the wing operating member is capable of being manually driven by an operator to move the wing driving member from the fourth position to the third position so as to fold the stabilizing wings.

In some embodiments, the wing operating member comprises a connecting arm having an engagement hook, the wing driving member is formed with a circumferential slot, the engagement hook fits into the circumferential slot, and the wing operating member, when rotating, is capable of driving the engagement hook to move relative to the circumferential slot.

In a third aspect, the present invention provides an operating method of a tissue suturing device. The operating method comprises a wing driving phase, a transition phase, and a needle driving phase which are sequentially implemented, wherein in the wing driving phase, a wing operating member of a tissue suturing device is driven to move axially from a third position to a fourth position, so as to unfold stabilizing wings of the tissue suturing device; in the transition phase, the wing operating member is rotated to drive a needle operating member of the tissue suturing device to rotate by a predetermined angle, such that the wing operating member is locked to a tube body of the tissue suturing device to prevent rotation and axial movement of the wing operating member so as to be maintained in the fourth position, and the needle operating member is unlocked from the tube body; and in the needle driving phase, the needle operating member is driven to move axially from the first position to a second position, so as to drive suturing needles to extend out of the tube body of the tissue suturing device to a puncture position.

In this way, in the wing driving phase, only the wing operating member can be driven to move axially, and the needle operating member cannot be driven to move axially. In this case, even if the needle operating member is incorrectly operated or accidentally touched, the needle operating member cannot drive the needle mounting member to move axially, and the suturing needles mounted on the needle mounting member thus cannot extend out of the tube body. In this way, accidental extension of the suturing needles can be completely avoided, thereby significantly improving the safety of using the tissue suturing device. The transition path can allow the needle operating member to move axially only. In this way, in the needle driving phase, the needle operating member can move axially from the first position to the second position, such that the suturing needles extend out of the tube body.

In some embodiments, a tactile feedback can be provided when the wing operating member is locked to and unlocked from the tube body.

In some embodiments, after the wing operating member is reversely rotated to unlock the wing operating member from the tube body, the wing operating member is capable of being manually driven by an operator to move reversely to the third position, and/or, the wing operating member is capable of automatically moving reversely to the third position under the action of energy released by an elastic member, so as to fold the stabilizing wings.

In some embodiments, the wing operating member and the needle operating member are locked by means of a same locking part of the tube body.

In a fourth aspect, the present invention provides a tissue suturing device capable of implementing the operating method of a tissue suturing device according to any of the foregoing described in the third aspect above. In this way, as described above, the tissue suturing device can completely avoid accidental extension of the suturing needle, thereby significantly improving the safety of using the tissue suturing device.

It will be apparent that elements or features described in each of the above embodiments may be used individually or in combination in other embodiments.

### BRIEF DESCRIPTION OF DRAWINGS

In the drawings, the dimensions and proportions do not represent those of actual products. The drawings are illustrative only and certain non-essential elements or features are omitted for the sake of clarity.

FIG. 1 is a perspective view schematically showing a tissue suturing device according to an embodiment of the present invention from one angle of view.

FIG. 2 is a perspective view in which a half tube of the tissue suturing device in FIG. 1 is omitted to clearly show an operating assembly.

FIG. 3 is a schematic diagram in which the structure of FIG. 1 is partially enlarged.

FIG. 4 is a schematic structural diagram in which a part of a wing operating member, a part of a tube body, and a part of a locking block on the tube body in FIG. 3 are omitted to clearly show that in an initial position, the locking block on the tube body fits into a first distal axial slot and a second distal axial slot.

FIG. 5 is a perspective view of a needle operating member of the operating assembly of the tissue suturing device in FIG. 1.

FIG. 6 is a perspective view of a wing operating member of the operating assembly of the tissue suturing device in FIG. 1.

FIG. 7 is a partial schematic structural diagram of a half tube of the tube body of the tissue suturing device in FIG. 1.

FIG. 8 is a schematic structural diagram showing cooperation between a wing operating member and a wing driving member of the tissue suturing device in FIG. 1.

FIG. 9 is a schematic structural diagram showing cooperation between a needle operating member and a needle mounting member of the tissue suturing device in FIG. 1.

FIG. 10 is a perspective view in which the tissue suturing device of FIG. 1 completes a wing driving phase.

FIG. 11 is a schematic partial structural view of FIG. 10, in which a half tube is moved away to clearly show an internal structure.

FIG. 12 is a perspective view in which the tissue suturing device of FIG. 1 completes a needle driving phase.

FIG. 13 is a schematic partial structural view of FIG. 12, in which a half tube is moved away to clearly show an internal structure.

FIG. 14 is a schematic structural diagram showing cooperation among a needle operating member, a wing driving member, a needle mounting member, and suturing needles of the tissue suturing device in FIG. 1.

### DESCRIPTION OF REFERENCE SIGNS

1—tissue suturing device, 2—tube body, 3—proximal end, 4—distal end, 5—axial channel, 6—wing operating member, 7—needle operating member, 8—axial sidewall, 9—locking block, 10—grip part, 11—locking protrusion, 12—locking recess, 13—guide slot, 14—guide block, 15—proximal slot wall, 16—first proximal axial slot, 17—first distal axial slot, 18—first stopper platform, 19—second proximal axial slot, 20—second distal axial slot, 21—second stopper platform, 22—half tube, 23—wing driving member, 24—stabilizing wing, 25—needle mounting member, 26—suturing needle, 27—elastic member, 28—elongated rod, 29—connecting arm, 30—circumferential slot, and 31—insertion block.

### DETAILED DESCRIPTION

The present invention will be described in detail below with reference to the accompanying drawings. The embodiments described herein are merely preferred embodiments according to the present invention, and on the basis of the preferred embodiments, those skilled in the art could conceive of other modes capable of implementing the present invention, which also fall within the scope of the present invention.

The proximal end mentioned in the present invention refers to an end of a tissue suturing device adjacent to or facing the operator when in use, and the distal end refers to an end of the tissue suturing device located away from or facing away from the operator when in use.

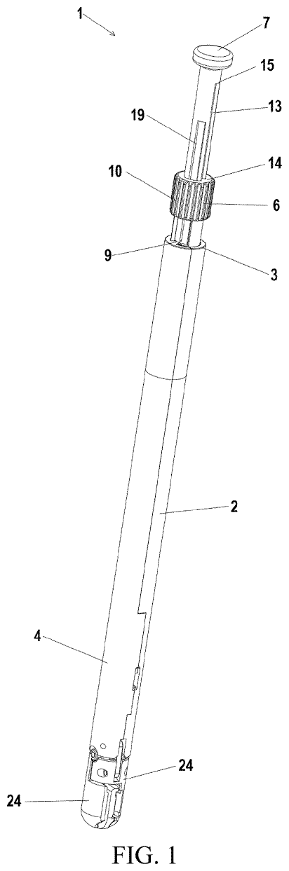

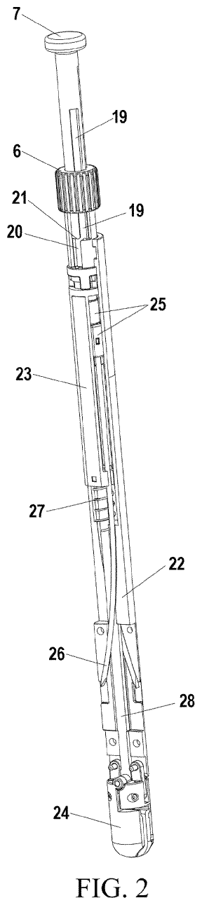

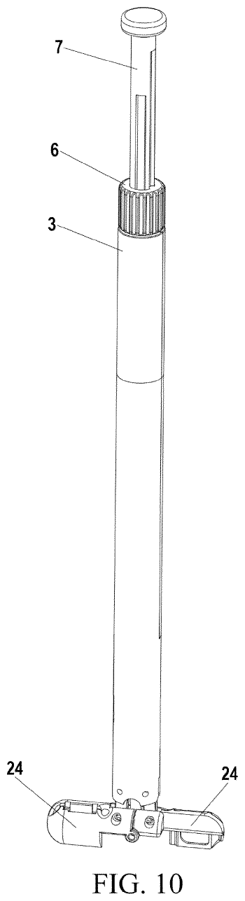

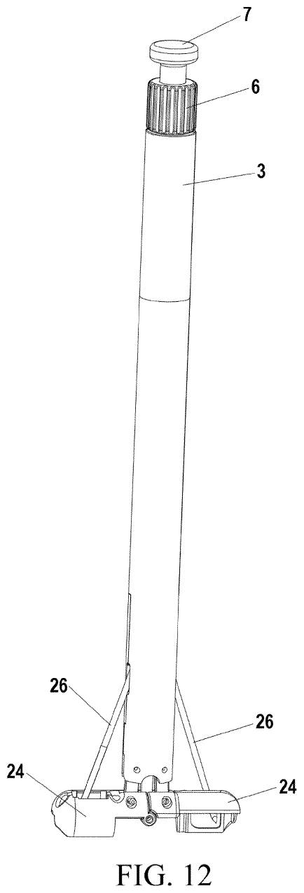

In a first aspect, referring to FIG. 1, FIG. 2, FIG. 10, and FIG. 12, the present invention provides an operating assembly for a tissue suturing device 1. The operating assembly includes a tube body 2, a wing operating member 6, and a needle operating member 7. The tube body 2 includes a proximal end 3, a distal end 4, and an axial channel 5 extending between the proximal end 3 and the distal end 4; the wing operating member 6 is axially and movably installed at the axial channel and is rotatable; and the needle operating member 7 is arranged in the wing operating member 6 and is rotatable along with the wing operating member 6 and axially movable relative to the wing operating member 6. The operating assembly includes an initial position, and a wing driving phase, a transition phase, and a needle driving phase which are sequentially implemented. In the initial position, the needle operating member 7 and the tube body 2 are in a first locking state, in which the needle operating member 7 is prevented from moving axially towards the distal end 4 to maintain the needle operating member 7 in a first position, the first position being used to enable suturing needles to be in a retracted state, and the wing operating member 6 and the tube body 2 are in a second locking state, in which the wing operating member 6 is prevented from rotating and is allowed to move axially towards the distal end 4 so as to maintain the wing operating member 6 in a third position, the third position being used to enable stabilizing wings to be in a folded state. Because the wing operating member 6 is non-rotatable in the second locking state, the needle operating member 7 is also non-rotatable in the first locking state, such that the needle operating member 7 is maintained in the first position. In the wing driving phase, the wing operating member 6 moves axially towards the distal end 4 from the third position to a fourth position used to enable the stabilizing wings to be in an unfolded state, and at the time, the needle operating member 7 is still maintained in the first position. In the transition phase, the wing operating member 6 drives the needle operating member 7 to rotate by a predetermined angle, such that the wing operating member 6 and the tube body 2 are in a third locking state in which rotation and axial movement of the wing operating member 6 are prevented, so as to be maintained in the fourth position; in the third locking state, the wing operating member 6 cannot move axially towards the proximal end or the distal end, such that the stabilizing wings can be stably and reliably maintained in the unfolded state during actual use; and the needle operating member 7 and the tube body 2 are unlocked from the first locking state to be axially movable only towards the distal end 4. In the needle driving phase, the needle operating member 7 moves axially towards the distal end 4 from the first position to a second position, the second position being used to enable the suturing needles to be in an extended state or a puncture position.

In this technical solution, the operating assembly includes the initial position, and the wing driving phase, transition phase, and needle driving phase which are sequentially implemented, and the wing operating member 6 is configured to drive a wing driving member to move axially towards the distal end 4 only in the wing driving phase, while the needle operating member 7 is configured to drive a needle mounting member to move axially towards the distal end 4 only in the needle driving phase. As a result, during actual use, in the wing driving phase, only the wing operating member 6 can be driven to move axially towards the distal end 4, and the needle operating member 7 cannot be driven to move axially towards the distal end 4. In this case, even if the needle operating member 7 is incorrectly operated or accidentally touched, the needle operating member 7 cannot drive the needle mounting member to move axially towards the distal end 4, and the suturing needles mounted on the needle mounting member thus cannot extend out of the tube body. In this way, accidental extension of the suturing needles can be completely avoided, thereby significantly improving the safety of using the tissue suturing device. By means of the transition phase, the needle operating member 7 and the tube body 2 can be unlocked to be axially movable only towards the distal end 4. In this way, in the needle driving phase, the needle operating member can move axially from the first position to the second position, such that the suturing needles extend out of the tube body. In addition, in some embodiments, a wing driving member 23 (described below) rotatably connected to the wing operating member 6 can only move in the axial direction of the tube body 2, and at the time, the wing operating member 6 in the third locking state cannot move axially towards the distal end 4, As a result, the wing driving member 23 at the time also cannot move axially towards the distal end 4, and is thus stably and reliably in the fourth position to stably and reliably maintain the stabilizing wings in an unfolded position, thereby avoiding accidental folding of the stabilizing wings, and further improving the reliability of the operating assembly. In addition, when retraction of the suturing needles and folding of the stabilizing wings are required, the needle operating member 7 is moved axially towards the proximal end 3 from the second position to the first position to retract the suturing needles, then the wing operating member 6 is reversely rotated to drive the needle operating member 7 to reversely rotate by a predetermined angle, then the wing operating member 6 is moved axially towards the proximal end 3 from the fourth position to the third position to fold the stabilizing wings, and finally the tissue suturing device is removed from tissues. Hence, the operations simply need to be reversed in sequence.

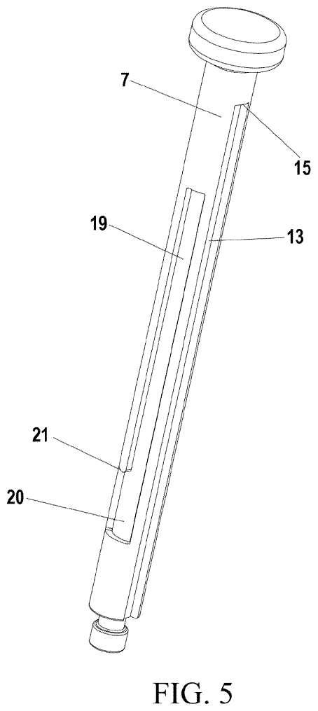

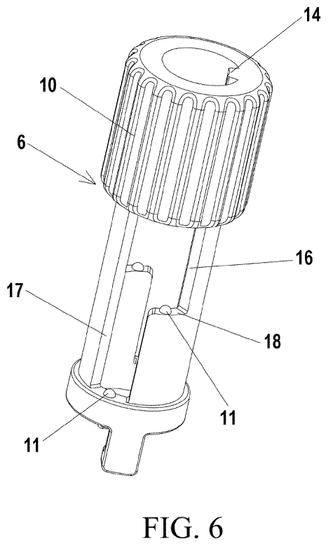

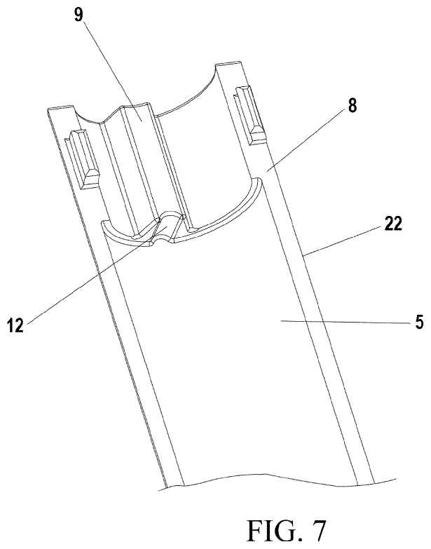

In addition, in the operating assembly of the present invention, in some embodiments, locking in the second locking state, the first locking state, and the third locking state can be implemented by means of locking parts at different positions on the tube body 2, respectively. In some other embodiments, referring to FIG. 5, FIG. 6, and FIG. 7, in at least two locking states among the second locking state, the first locking state, and the third locking state, locking is implemented based on the same locking part of the tube body 2. In this way, the same locking part of the tube body 2 can be fully utilized to implement locking in multiple locking states, enabling the operating assembly to have a compact structure, and avoiding forming multiple different locking parts on the tube body 2.

In addition, in some embodiments, in the first locking state, the second locking state, and the third locking state, locking is implemented based on the same locking part, that is, the wing operating member 6 and the needle operating member 7 are locked by means of the same locking part, which can further enable the operating assembly to have a compact structure.

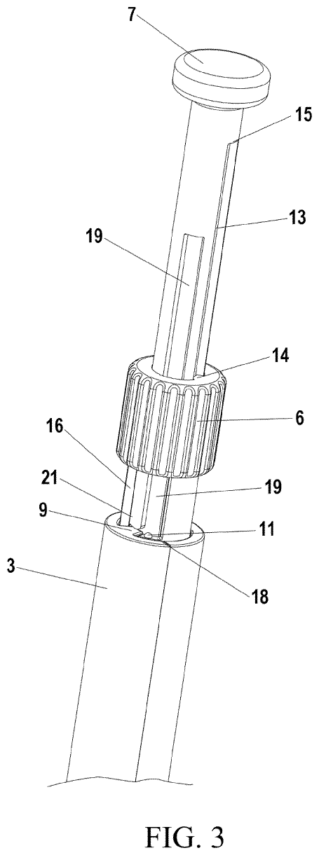

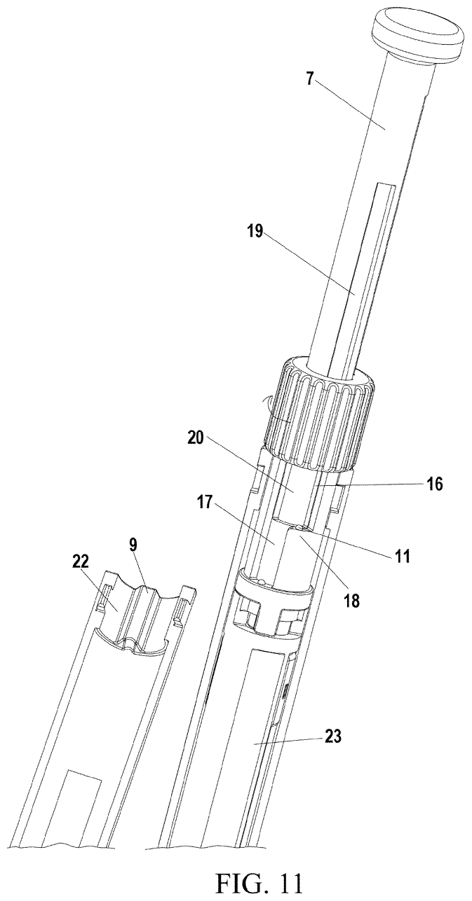

Certainly, the locking part may have a variety of types. No matter which type is used, said type is acceptable as long as the locking part can implement the locking states described above. For example, referring to FIG. 7, the locking part is a locking block 9 extending inwards radially from an inside surface of an axial sidewall 8 of the tube body 2. In this way, the wing operating member 6 and the needle operating member 7 can be locked by means of a single locking block 9. Referring to FIG. 1 and FIG. 3, the wing operating member 6 abuts against a circumferential side surface of the locking block 9, such that the wing operating member 6 and the tube body 2 are in the second locking state in which the wing operating member 6 is prevented from rotating; moreover, the needle operating member 7 abuts against a proximal end surface of the locking block 9, such that the needle operating member 7 and the tube body 2 are in the first locking state in which the needle operating member 7 is prevented from moving axially towards the distal end 4. Referring to FIG. 7, FIG. 10, and FIG. 11, after completing the wing driving phase and the transition phase, the wing operating member 6 abuts against a distal end surface of the locking block 9, such that the wing operating member 6 and the tube body 2 are in the third locking state in which rotation and axial movement of the wing operating member 6 are prevented. In the third locking state, the wing operating member 6 is non-rotatable unless a reverse rotating force applied by an operator is greater than a locking force between the wing operating member 6 and the distal end surface of the locking block 9, and the wing operating member 6 cannot move axially towards the proximal end or the distal end. Thus, it can be seen that the three locking states can be achieved only by one locking block 9.

In addition, in some embodiments, in order to make the operator easily recognize the completion of the wing driving phase and to make the wing operating member 6 accurately stop in the fourth position, with reference to FIG. 10 and FIG. 11, in the fourth position, an end surface of the proximal end 3 is in contact with a distal end surface of a grip part 10 of the wing operating member 6 to stop the wing operating member 6 from moving axially towards the distal end 4. In this way, when the operator observes that the distal end surface of the grip part 10 abuts against the end surface of the proximal end 3, the wing driving stage is recognized as completed.

In addition, in some embodiments, in order to make the operator readily recognize the completion of the transition phase, a locking protrusion 11 is formed on one of the wing operating member 6 and the tube body 2, and a locking recess 12 is formed on the other of the wing operating member and the tube body, and when the wing operating member 6 rotates to drive the locking protrusion 11 to be pressed into or out of the locking recess 12, a tactile feedback (such as a clicking sound) of being in or out of the third locking state is provided. For example, when a clicking sound occurs, the transition phase is recognized as completed, the wing operating member 6 is in the third locking state, and the needle operating member 7 is unlocked from the tube body 2 to be axially movable only towards the distal end 4. For example, in the embodiments shown in FIG. 6 and FIG. 7, the locking protrusion 11 is formed on the wing operating member 6, and the locking recess 12 is formed on the distal end surface of the locking block 9 of the tube body 2. In this way, when the operator does not apply a reverse rotating force, the locking protrusion 11 is maintained in the locking recess 12. When the wing operating member 6 needs to be reversely rotated, the reverse rotating force applied by the operator is greater than a locking force between the locking protrusion 11 and the locking recess 12, such that the locking protrusion 11 can be separated from the locking recess 12 to reversely rotate the wing operating member 6.

In addition, in some embodiments, referring to FIG. 3, FIG. 5, and FIG. 6, an axially extending guide slot 13 is formed on one of the needle operating member 7 and the wing operating member 6, and a guide block 14 is formed on the other of the needle operating member and the wing operating member. The guide block 14 fits into the guide slot 13, such that the needle operating member 7 is rotatable along with the wing operating member 6 and axially movable relative to the wing operating member 6. In this way, when the wing operating member 6 rotates, the needle operating member 7 can be driven to rotate, such that the needle operating member 7 is unlocked from the tube body 2 to be axially movable in the needle driving phase.

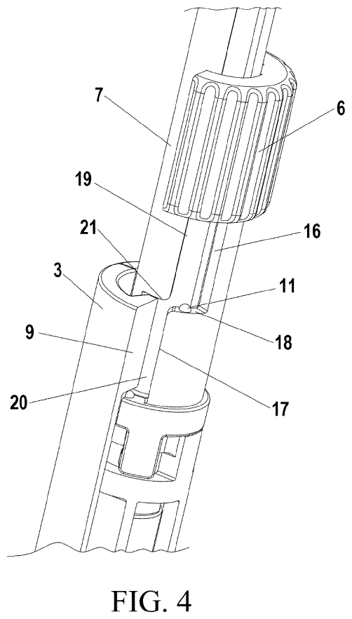

In addition, in some embodiments, in order to enable the suturing needles to accurately extend to the puncture position, with reference to FIG. 3 to FIG. 6, in the second position, a proximal slot wall 15 of the guide slot 13 is in contact with the guide block 14 to stop the needle operating member 7 from moving axially towards the distal end 4.

In addition, in the operating assembly of the present invention, the wing operating member 6 may have a variety of structures. No matter which structure is used, said structure is acceptable as long as the effects described above can be achieved. For example, in some embodiments, referring to FIG. 6, a first proximal axial slot 16 and a first distal axial slot 17 communicating with each other are formed on an outer peripheral surface of the wing operating member 6, a circumferential sidewall of the first proximal axial slot 16 is aligned with a circumferential sidewall of the first distal axial slot 17, and the circumferential dimension of the first proximal axial slot 16 is greater than the circumferential dimension of the first distal axial slot 17, so that a first stopper platform 18 is formed therebetween. In this way, after the locking part of the tube body 2 enters the first proximal axial slot 16 from the first distal axial slot 17, due to the greater circumferential dimension of the first proximal axial slot 16, the wing operating member 6 can be allowed to rotate in the transition phase, such that the locking part of the tube body 2, in the first proximal axial slot 16, is located between the first stopper platform 18 and the proximal slot wall of the first proximal axial slot 16. In the initial position, the locking part (such as the locking block 9) of the tube body 2 fits into the first distal axial slot 17, and at the time, the wing operating member 6 can be prevented from rotating due to the circumferential limiting effect of the first distal axial slot 17 on the locking block 9. The locking part such as the locking block 9 is axially aligned with the first proximal axial slot 16 to enable the wing operating member 6 to move axially towards the distal end 4, such that the wing operating member 6 is non-rotatable, and the wing operating member 6 and the tube body (2) are in the second locking state. In the wing driving phase, the wing operating member 6 is driven by the operator to move axially towards the distal end 4, such that the locking part enters the first proximal axial slot 16 from the first distal axial slot 17, and at the time, due to the fact that the circumferential dimension of the first proximal axial slot 16 is greater than the circumferential dimension of the first distal axial slot 17, the wing operating member 6 is rotatable and simultaneously drives the needle operating member 7 to rotate. In the transition phase, after the wing operating member 6 rotates by a predetermined angle, the locking part in the first proximal axial slot 16 is located between the first stopper platform 18 and the proximal slot wall of the first proximal axial slot 16, and at the time, the locking part is locked to the first stopper platform 18, such that the wing operating member 6 and the tube body 2 are in the third locking state, and the wing operating member 6 is non-rotatable and cannot move axially towards the proximal end or the distal end. In this way, in some embodiments, the wing driving member 23 (described below) rotatably connected to the wing operating member 6 can only move in the axial direction of the tube body 2, and at the time, the wing operating member 6 in the third locking state cannot move axially towards the proximal end or the distal end. As a result, the wing driving member 23 at the time also cannot move axially towards the proximal end or the distal end, and is thus stably and reliably in the fourth position to stably and reliably maintain the stabilizing wings in the unfolded position, thereby avoiding accidental folding of the stabilizing wings, and further improving the reliability of the operating assembly.

In addition, in some embodiments, a locking recess 12 is formed on one of a surface of the first stopper platform 18 and a distal end surface of the locking part, and a locking protrusion 11 is formed on the other of the surface of the first stopper platform and the distal end surface of the locking part. When the wing operating member 6 rotates to drive the locking protrusion 11 to be pressed into or out of the locking recess 12, the tactile feedback (such as a clicking sound) of being in or out of the third locking state is provided. For example, when a clicking sound occurs, the transition phase is recognized as completed, the wing operating member 6 is in the third locking state, and the needle operating member 7 is unlocked from the tube body 2 to be axially movable only.

In addition, in the operating assembly of the present invention, the needle operating member 7 may have a variety of structures. No matter which structure is used, said structure is acceptable as long as the effects described above can be achieved. For example, in some embodiments, referring to FIG. 5, a second proximal axial slot 19 and a second distal axial slot 20 communicating with each other are formed on an outer peripheral surface of the needle operating member 7, and the circumferential dimension of the second proximal axial slot 19 is less than the circumferential dimension of the second distal axial slot 20, such that a second stopper platform 21 is formed therebetween. In the initial position, a locking part of the tube body 2 fits into the second distal axial slot 20 and is engaged with the second stopper platform 21, such that the needle operating member 7 and the tube body 2 are in the first locking state, and at the time, the needle operating member 7 cannot move axially. In the transition phase, the wing operating member 6 drives the needle operating member 7 to rotate by a predetermined angle, such that the locking part is separated from the second stopper platform 21 and axially aligned with the second proximal axial slot 19 to unlock the needle operating member 7 from the tube body 2, and the needle operating member 7 can be allowed to move axially. In the needle driving phase, the locking part and the second proximal axial slot 19 are axially movable relative to each other.

In addition, in some embodiments, in order to improve the convenience of installation and easy processing of the operating assembly, with reference to FIG. 1 and FIG. 2, the tube body 2 includes two half tubes 22 capable of being joined together; and each of the half tubes 22 is capable of being in the second locking state and the third locking state together with the wing operating member 6 and being in the first locking state together with the needle operating member 7. In this way, the operational balance between the wing operating member 6 and the needle operating member 7 can be effectively improved.

In one embodiment, referring to FIG. 3, in the initial position, the locking block 9 fits into the first distal axial slot 17 and the second distal axial slot 20, a circumferential side surface of the locking block 9 abuts against a circumferential side surface of the first distal axial slot 17 to prevent rotation of the wing operating member 6, and at the time, the needle operating member 7 is also non-rotatable, and the second stopper platform 21 abuts against the proximal end surface of the locking block 9 to prevent axial movement of the needle operating member 7. In the wing driving phase, the wing operating member 6 moves axially, the locking block 9 enters the first proximal axial slot 16 from the first distal axial slot 17, and due to the fact that the circumferential dimension of the first proximal axial slot 16 is greater than the circumferential dimension of the first distal axial slot 17, the wing operating member 6 is rotatable, such that the locking protrusion 11 on the surface of the first stopper platform 18 enters the locking recess 12 on the distal end surface of the locking block 9 to form the tactile feedback of a clicking sound. At the time, the wing operating member 6 is non-rotatable and cannot move axially, so as to be maintained in the fourth position. While rotating, the wing operating member 6 simultaneously drives the needle operating member 7 to rotate, such that the proximal end surface of the locking block 9 is separated from the second stopper platform 21 and is axially aligned with the second proximal axial slot 19. In the needle driving phase, the needle operating member 7 moves axially to extend the suturing needles. During retraction of the suturing needles and folding of the stabilizing wings, the operations simply need to be performed reversely.

In addition, in some embodiments, referring to FIG. 6, a locking protrusion 11 may also be formed on a slot bottom wall of the first distal axial slot 17 of the wing operating member 6. In this way, when the locking block 9 is located in the first distal axial slot 17, the locking protrusion 11 fits with the locking recess 12 in the locking block 9, thereby avoiding circumferential shaking of the wing operating member 6 when in the initial position. In this way, an allowed circumferential gap can be formed between the first distal axial slot 17 and the locking block 9, making it easier for the operator to push the wing operating member 6 towards the distal end 4.

The operating process of the operating assembly shown in the drawings is described in detail below with reference to FIGS. 1 to 13. FIG. 1 to FIG. 4 show the operating assembly in the initial position. In the initial position, the locking block 9 on the tube body 2 fits into the first distal axial slot 17 of the wing operating member 6 to limit rotation of the wing operating member 6, and the locking block 9 is axially aligned with the first proximal axial slot 16 to enable the wing operating member 6 to move axially towards the distal end 4. Moreover, the locking block 9 extends inwards radially to fit into the second distal axial slot 20 of the needle operating member 7 and be in contact with the second stopper platform 21. Due to the stoppage of the second stopper platform 21 by the locking block 9, the needle operating member 7 cannot move axially towards the distal end 4. In this case, because the wing operating member 6 is non-rotatable, the operating member 7 is also non-rotatable. In this way, in the initial position, only the wing operating member 6 can move axially towards the distal end 4, the stabilizing wings are in the retracted state, and the suturing needles are within the tube body.

In the wing driving phase, as shown in FIGS. 10 to 11, the operator moves the wing operating member 6 axially towards the distal end 4, such that the locking block 9 enters the first proximal axial slot 16 from the first distal axial slot 17, so as to drive the stabilizer wings 24 to be unfolded. At the time, the locking block 9 is separated from the first distal axial slot 17. As a result, the wing operating member 6 is rotatable, and the locking block 9 is not in contact with the first stopper platform 18.

In the transition phase, the wing operating member 6 is rotated by the predetermined angle along a rotation arrow shown in FIG. 11, such that when the locking block 9 moves in the first proximal axial slot 16 to the position between the first stopper platform 18 and the proximal slot wall (such as the distal end surface of the grip part 10 of the wing operating member 6) of the first proximal axial slot 16, the locking protrusion 11 on the first stopper platform 18 enters the locking recess 12 in the distal end surface of the locking block 9. For example, a tactile feedback (such as a clicking sound) is provided. At the time, the operator can recognize that the transition phase is completed. Moreover, while rotating, the wing operating member 6 simultaneously drives the needle operating member 7 to perform synchronous rotation, such that the locking block 9 in the second distal axial slot 20 moves to the position where the locking block is axially aligned with the second proximal axial slot 19 and separated from the second stopper platform 21. In this case, the needle operating member 7 can move axially towards the distal end 4. Therefore, it can be seen that the predetermined angle is an angle by which the wing operating member 6 is rotated to move the locking block 9 from the second distal axial slot 20 to be axially aligned with the second proximal axial slot 19 to enable the needle operating member 7 to move axially towards the distal end.

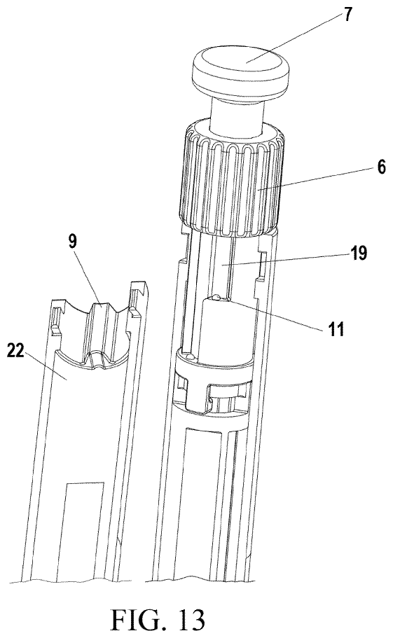

In the needle driving phase, as shown in FIG. 12 and FIG. 13, due to the fact that the locking block 9 is axially aligned with the second proximal axial slot 19 and separated from the second stopper platform 21, the operator can drive the needle operating member 7 to move towards the distal end 4, such that the locking block 9 moves into the second proximal axial slot 19, thereby extending the suturing needles to the puncture position.

When retraction of the suturing needles and folding of the stabilizing wings are required, the operations simply need to be performed reversely.

In a second aspect, the present invention provides a tissue suturing device 1. Referring to FIG. 1 to FIG. 14, the tissue suturing device 1 includes the operating assembly according to any of the foregoing described in the first aspect above, a wing driving member 23, stabilizing wings 24, a needle mounting member 25, and suturing needles 26. The wing driving member 23 is axially and movably arranged in the axial channel; the stabilizing wings 24 are hinged to the distal end 4 of the tube body 2 and hinged to the wing driving member 23; the needle mounting member 25 is axially and movably arranged in the axial channel; and the suturing needles 26 are arranged on the needle mounting member 25 and located in the tube body 2. The wing operating member 6 is rotatably connected to the wing driving member 23, and the needle operating member 7 is rotatably connected to the needle mounting member 25. The wing operating member 6 is capable of driving, by means of the wing driving member 23, the stabilizing wings 24 to be unfolded and folded, and the needle operating member 7 is capable of driving, by means of the needle mounting member 25, the suturing needles 26 to extend out of the tube body 2 to a puncture position and to retract back into the tube body 2. In this way, as described in the first aspect above, by means of the operating assembly, the tissue suturing device can completely avoid accidental extension of the suturing needle, thereby significantly improving the safety of using the tissue suturing device.

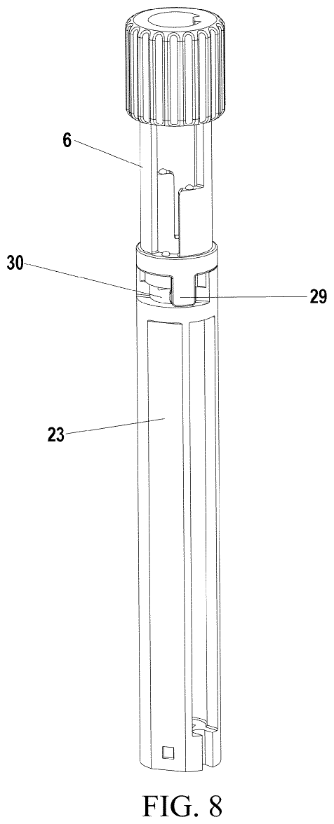

The rotatable connection between the wing operating member 6 and the wing driving member 23 can be implemented in various forms. No matter which form is used, said form is acceptable as long as the rotatable connection can be implemented. For example, in one form, the wing operating member and the wing driving member may be connected by a bearing. In another form, referring to FIG. 8, the wing operating member 6 includes a connecting arm 29 having an engagement hook, the wing driving member 23 is formed with a circumferential slot 30, the engagement hook fits into the circumferential slot 30, and the wing operating member 6, when rotating, is capable of driving the engagement hook to move relative to the circumferential slot 30. In this way, the rotatable connection between the wing operating member 6 and the wing driving member 23 can be implemented. In addition, in some embodiments, one or more connecting arms 29 are formed at a distal end of the wing operating member 6, and one or more circumferential slots 30 are formed at a proximal end of the wing driving member 23. The circumferential slot 30 may be a circumferential slot section or may be a complete circular slot. An engagement hook at a front end of the connecting arm 29 can fit into the circumferential slot 30. In this way, in the wing driving phase, the wing operating member 6 can drive the wing driving member 23 to move axially in a forward or reverse direction to unfold or fold the stabilizing wings. Moreover, in the transition phase, the wing operating member 6 is rotatable relative to the wing driving member 23. For example, after the wing operating member 6 is reversely rotated to unlock the wing operating member 6 from the tube body 2, that is, after the wing operating member and the tube body are unlocked from the third locking state, due to the fact that an engagement hook at a distal end of the connecting arm 29 of the wing operating member 6 fits into the circumferential slot 30 of the wing driving member 23, the operator can manually drive the wing operating member 6 to move reversely towards the proximal end of the tissue suturing device, and drive the wing driving member 23 to perform corresponding reverse movement, such that the stabilizing wings are rotated from the fourth position to the third position and are reliably folded.

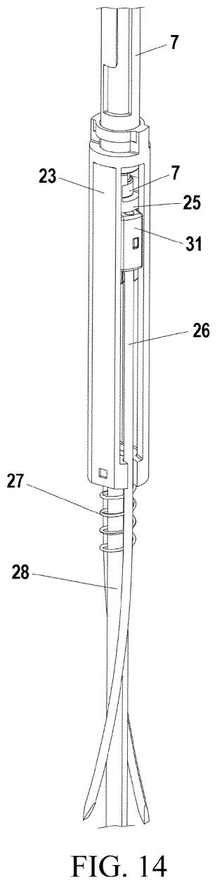

In addition, in some embodiments, referring to FIG. 14, an elongated rod 28 may be connected to a distal end of the wing driving member 23, and may be hinged to the stabilizing wings.

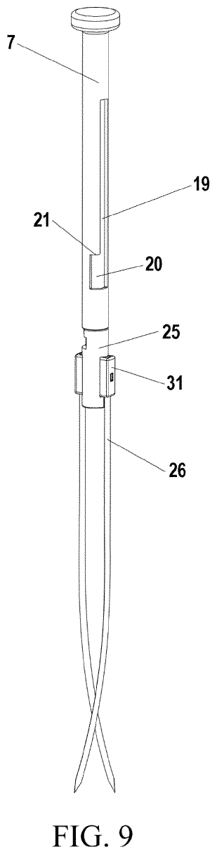

In addition, the rotatable connection between the needle operating member 7 and the needle mounting member 25 can be implemented in various forms. No matter which form is used, said form is acceptable as long as the rotatable connection can be implemented. For example, in one form, the needle operating member and the needle mounting member may be connected by a bearing. In another form, referring to FIG. 5 and FIG. 9, a connector such as a cylindrical head or a spherical head is formed at a distal end of the needle operating member 7, a connecting recess is formed at a proximal end of the needle mounting member 25, and the cylindrical head or the spherical head rotatably fits into the connecting recess. In addition, in some embodiments, the suturing needles can be directly mounted on the needle mounting member 25. Alternatively, in some other embodiments, referring to FIG. 9, insertion blocks 31 are arranged on the needle mounting member 25, and the suturing needles can be mounted in the insertion blocks 31.

In addition, in some embodiments, referring to FIG. 14, the wing driving member 23 is formed with an axially extending hollow channel, the needle mounting member 25 is arranged in the hollow channel in an axially movable manner, and the needle operating member 7 is connected to the needle mounting member 25 and is capable of extending axially into the hollow channel. In this way, the hollow channel of the wing driving member 23 can provide guidance for the axial movement of the needle operating member 7 and the needle mounting member 25, such that the needle operating member 7 and the needle mounting member 25 can stably perform axial movement, thereby effectively improving the stability of extending the suturing needles.

In addition, referring to FIG. 14, an elastic member 27 is arranged between the wing driving member 23 and the tube body 2. When the wing operating member 6 drives the wing driving member 23 to move axially from the third position to the fourth position, the elastic member 27 stores energy; and when the wing operating member 6 and the tube body 2 are unlocked from the third locking state, the elastic member 27 releases energy to drive, by means of the wing driving member 23, the wing operating member 6 to return from the fourth position to the third position. In this way, the operator can easily fold the stabilizing wings under an elastic restoring force provided by the elastic member 27.

In addition, when the elastic member 27 fails or malfunctions, the wing operating member 6 is capable of being manually driven by the operator to move the wing driving member 23 from the fourth position to the third position so as to fold the stabilizing wings 24. For example, in some embodiments, when the elastic member 27 malfunctions, due to the rotatable connection between the wing operating member 6 and the wing driving member 23 (for example, in some embodiments, the engagement hook at the front end of the connecting arm 29 fits into the circumferential slot 30), the operator can manually drive the wing operating member to reversely move to the third position, so as to fold the stabilizing wings. For example, after the wing operating member 6 is reversely rotated to unlock the wing operating member 6 from the tube body 2, that is, after the wing operating member and the tube body are unlocked from the third locking state, if the elastic force 27 malfunctions or fails and cannot provide the elastic restoring force, due to the fact that the engagement hook at the distal end of the connecting arm 29 of the wing operating member 6 fits into the circumferential slot 30 of the wing driving member 23, the operator can manually drive the wing operating member 6 to move reversely towards the proximal end of the tissue suturing device, and drive the wing driving member 23 to perform corresponding reverse movement, such that the stabilizing wings are rotated from the fourth position to the third position and are reliably folded.

In a third aspect, the present invention provides an operating method of a tissue suturing device. The operating method includes a wing driving phase, a transition phase, and a needle driving phase which are sequentially implemented, where in the wing driving phase, a wing operating member of a tissue suturing device is driven to move axially from a third position to a fourth position, so as to unfold stabilizing wings of the tissue suturing device; in the transition phase, the wing operating member is rotated to drive a needle operating member of the tissue suturing device to rotate by a predetermined angle, such that the wing operating member is locked to a tube body of the tissue suturing device to prevent rotation and axial movement of the wing operating member so as to be maintained in the fourth position, and the needle operating member is unlocked from the tube body; and in the needle driving phase, the needle operating member is driven to move axially from a first position to a second position, so as to drive suturing needles to extend out of the tube body of the tissue suturing device to a puncture position.

In this way, in the operating method of a tissue suturing device, in the wing driving phase, only the wing operating member can be driven to move axially, and the needle operating member cannot be driven to move axially. In this case, even if the needle operating member is incorrectly operated or accidentally touched, the needle operating member cannot drive the needle mounting member to move axially, and the suturing needles mounted on the needle mounting member thus cannot extend out of the tube body. In this way, accidental extension of the suturing needles can be completely avoided, thereby significantly improving the safety of using the tissue suturing device. The transition path can allow the needle operating member to move axially only. In this way, in the needle driving phase, the needle operating member can move axially from the first position to the second position, such that the suturing needles extend out of the tube body.

In addition, in some embodiments, a tactile feedback is can be provided when the wing operating member is locked to and unlocked from the tube body. In this way, an operator can easily recognize that the wing operating member rotates to drive a locking protrusion to be pressed into or out of a locking recess, so as to recognize at the earliest time that the wing operating member is in or out of the third locking state.

In addition, in some embodiments, after the wing operating member is reversely rotated to unlock the wing operating member from the tube body, the wing operating member is capable of being manually driven by the operator to move reversely to the third position, and/or, the wing operating member is capable of automatically moving reversely to the third position under the action of energy released by an elastic member, so as to fold the stabilizing wings. In this way, in some embodiments, the operator can easily fold the stabilizing wings with the assistance of the elastic member. In some other embodiments, if the elastic member malfunctions, the operator can manually drive the wing operating member to move reversely to the third position, so as to fold the stabilizing wings. For example, in some embodiments, an engagement hook at a front end of a connecting arm 29 fits into a circumferential slot 30, such that the operator can manually drive the wing operating member to move reversely to the third position, so as to fold the stabilizing wings. For example, after the wing operating member 6 is reversely rotated to unlock the wing operating member 6 from the tube body 2, that is, after the wing operating member and the tube body are unlocked from the third locking state, if the elastic force 27 malfunctions or fails and cannot provide an elastic restoring force, due to the fact that the engagement hook at a distal end of the connecting arm 29 of the wing operating member 6 fits into the circumferential slot 30 of the wing driving member 23, the operator can manually drive the wing operating member 6 to move reversely, and drive the wing driving member 23 to perform corresponding reverse movement, such that the stabilizing wings are rotated from the fourth position to the third position and are reliably folded.

In addition, the wing operating member and the needle operating member are locked by means of the same locking part of the tube body. In this way, the same locking part of the tube body can be fully utilized to implement locking and unlocking of the wing operating member and the needle operating member, enabling the tissue suturing device to have a compact structure, and avoiding forming multiple different locking parts on the tube body.

In a fourth aspect, the present invention provides a tissue suturing device capable of implementing the operating method of a tissue suturing device according to any of the foregoing described in the third aspect above. In this way, as described above, the tissue suturing device can completely avoid accidental extension of the suturing needle, thereby significantly improving the safety of using the tissue suturing device.

The scope of protection of the present invention is defined only by the claims. In light of the teachings of the present invention, those skilled in the art would readily recognize that alternative structures of the structures disclosed in the present invention may be used as feasible alternative embodiments, and the embodiments disclosed in the present invention may be combined to form new embodiments, which also fall within the scope of the appended claims.

1. An operating assembly for a tissue suturing device (1), the operating assembly comprising:
a tube body (2), the tube body (2) comprising a proximal end (3), a distal end (4), and an axial channel (5) extending between the proximal end (3) and the distal end (4);
a wing operating member (6), the wing operating member (6) being axially and movably installed at the axial channel and being rotatable; and
a needle operating member (7), the needle operating member (7) being arranged in the wing operating member (6), and the needle operating member (7) being rotatable along with the wing operating member (6) and being axially movable relative to the wing operating member (6);
the operating assembly comprising an initial position, and a wing driving phase, a transition phase, and a needle driving phase which are sequentially implemented, wherein:
in the initial position, the needle operating member (7) and the tube body (2) are in a first locking state, in which the needle operating member (7) is prevented from moving axially towards the distal end (4) so as to maintain the needle operating member (7) in a first position, the first position being used to enable suturing needles to be in a retracted state, and the wing operating member (6) and the tube body (2) are in a second locking state, in which the wing operating member (6) is prevented from rotating and is allowed to move axially towards the distal end (4) so as to maintain the wing operating member (6) in a third position, the third position being used to enable stabilizing wings to be in a folded state;
in the wing driving phase, the wing operating member (6) moves axially towards the distal end (4) from the third position to a fourth position so as to be unlocked from the second locking state, the fourth position being used to enable the stabilizing wings to be in an unfolded state;
in the transition phase, after the wing operating member (6) drives the needle operating member (7) to rotate by a predetermined angle, the wing operating member (6) and the tube body (2) are in a third locking state in which rotation and axial movement of the wing operating member (6) are prevented, so as to be maintained in the fourth position, and the needle operating member (7) is unlocked from the first locking state to be axially movable only towards the distal end (4); and
in the needle driving phase, the needle operating member (7) moves axially towards the distal end (4) from the first position to a second position, the second position being used to enable the suturing needles to be in an extended state.

2. The operating assembly according to claim 1, wherein in at least two locking states among the first locking state, the second locking state, and the third locking state, locking is implemented based on the same locking part of the tube body (2).

3. The operating assembly according to claim 2, wherein in the first locking state, the second locking state, and the third locking state, locking is implemented based on the same locking part.

4. The operating assembly according to claim 2, wherein the locking part is a locking block (9) extending inwards radially from an inside surface of an axial sidewall (8) of the tube body (2).

5. The operating assembly according to claim 1, wherein in the fourth position, an end surface of the proximal end (3) is in contact with a distal end surface of a grip part (10) of the wing operating member (6) to stop the wing operating member (6) from moving axially towards the distal end (4).

6. The operating assembly according to claim 1, wherein a locking protrusion (11) is formed on one of the wing operating member (6) and the tube body (2), a locking recess (12) is formed on the other of the wing operating member and the tube body, and when the wing operating member (6) rotates to drive the locking protrusion (11) to be pressed into or out of the locking recess (12), a tactile feedback of being in or out of the third locking state is provided.

7. The operating assembly according to claim 1, wherein an axially extending guide slot (13) is formed on one of the needle operating member (7) and the wing operating member (6), a guide block (14) is formed on the other of the needle operating member and the wing operating member, and the guide block (14) fits into the guide slot (13), such that the needle operating member (7) is rotatable along with the wing operating member (6) and is axially movable relative to the wing operating member (6).

8. The operating assembly according to claim 7, wherein in the second position, a proximal slot wall (15) of the guide slot (13) is in contact with the guide block (14) to stop the needle operating member (7) from moving axially towards the distal end.

9. The operating assembly according to claim 1, wherein a first proximal axial slot (16) and a first distal axial slot (17) communicating with each other are formed on an outer peripheral surface of the wing operating member (6), and the circumferential dimension of the first proximal axial slot (16) is greater than the circumferential dimension of the first distal axial slot (17), so that a first stopper platform (18) is formed therebetween, wherein:
in the initial position, the locking part of the tube body (2) fits into the first distal axial slot (17) to prevent rotation of the wing operating member (6), and the locking part is axially aligned with the first proximal axial slot (16) to enable the wing operating member (6) to move axially towards the distal end, such that the wing operating member (6) and the tube body (2) are in the second locking state;
in the wing driving phase, the wing operating member (6) moves axially towards the distal end, such that the locking part enters the first proximal axial slot (16) from the first distal axial slot (17); and
in the transition phase, after the wing operating member (6) rotates by a predetermined angle, the locking part and the first stopper platform (18) are locked, such that the wing operating member (6) and the tube body (2) are in the third locking state.

10. The operating assembly according to claim 9, wherein a locking recess (12) is formed on one of a surface of the first stopper platform (18) and a distal end surface of the locking part, and a locking protrusion (11) is formed on the other of the surface of the first stopper platform and the distal end surface of the locking part, wherein when the wing operating member (6) rotates to drive the locking protrusion (11) to be pressed into or out of the locking recess (12), a tactile feedback of being in or out of the third locking state is provided.

11. The operating assembly according to claim 1, wherein a second proximal axial slot (19) and a second distal axial slot (20) communicating with each other are formed on an outer peripheral surface of the needle operating member (7), and the circumferential dimension of the second proximal axial slot (19) is less than the circumferential dimension of the second distal axial slot (20), so that a second stopper platform (21) is formed therebetween, wherein:
in the initial position, a locking part of the tube body (2) fits into the second distal axial slot (20) and is engaged with the second stopper platform (21), such that the needle operating member (7) and the tube body (2) are in the first locking state;
in the transition phase, the wing operating member (6) drives the needle operating member (7) to rotate by a predetermined angle, such that the locking part is separated from the second stopper platform (21) and is axially aligned with the second proximal axial slot (19) to unlock the needle operating member (7) and the tube body (2); and
in the needle driving phase, the locking part and the second proximal axial slot (19) are axially movable relative to each other.

12. The operating assembly according to claim 1, wherein the tube body (2) comprises two half tubes (22) capable of being joined together, wherein each of the half tubes (22) is capable of being in the second locking state and the third locking state together with the wing operating member (6), and each of the half tubes (22) is capable of being in the first locking state together with the needle operating member (7).

13. A tissue suturing device, comprising:
the operating assembly according to claim 1;
a wing driving member (23), the wing driving member (23) being axially and movably arranged in the axial channel;
stabilizing wings (24), the stabilizing wings (24) being hinged to the distal end (4) of the tube body (2) and being hinged to the wing driving member (23);
a needle mounting member (25), the needle mounting member (25) being axially and movably arranged in the axial channel; and
suturing needles (26), the suturing needles (26) being arranged on the needle mounting member (25) and being located in the tube body (2);
wherein the wing operating member (6) is rotatably connected to the wing driving member (23), and the needle operating member (7) is rotatably connected to the needle mounting member (25);
wherein the wing operating member (6) is capable of driving, by means of the wing driving member (23), the stabilizing wings (24) to be unfolded and folded, and the needle operating member (7) is capable of driving, by means of the needle mounting member (25), the suturing needles (26) to extend out of the tube body (2) to a puncture position and to retract back into the tube body (2).

14. The tissue suturing device according to claim 13, wherein the wing driving member (23) is formed with an axially extending hollow channel, wherein the needle mounting member (25) is arranged in the hollow channel in an axially movable manner, and the needle operating member (7) is connected to the needle mounting member (25) and is capable of extending axially into the hollow channel.

15. The tissue suturing device according to claim 13, wherein an elastic member (27) is arranged between the wing driving member (23) and the tube body (2), wherein:
when the wing operating member (6) drives the wing driving member (23) to move axially from the third position to the fourth position, the elastic member (27) stores energy; and
when the wing operating member (6) and the tube body (2) are unlocked from the third locking state, the elastic member (27) releases energy to drive, by means of the wing driving member (23), the wing operating member (6) to return from the fourth position to the third position.

16. The tissue suturing device according to claim 15, wherein when the elastic member (27) fails or malfunctions, the wing operating member (6) is capable of being manually driven by an operator to move the wing driving member (23) from the fourth position to the third position so as to fold the stabilizing wings (24).

17. The tissue suturing device according to claim 13, wherein the wing operating member (6) comprises a connecting arm (29) having an engagement hook, the wing driving member (23) is formed with a circumferential slot (30), the engagement hook fits into the circumferential slot (30), and the wing operating member (6), when rotating, is capable of driving the engagement hook to move relative to the circumferential slot (30).

18. An operating method of a tissue suturing device, the operating method comprising a wing driving phase, a transition phase, and a needle driving phase which are sequentially implemented, wherein:
in the wing driving phase, a wing operating member of a tissue suturing device is driven to move axially from a third position to a fourth position, so as to unfold stabilizing wings of the tissue suturing device;
in the transition phase, the wing operating member is rotated to drive a needle operating member of the tissue suturing device to rotate by a predetermined angle, such that the wing operating member is locked to a tube body of the tissue suturing device to prevent rotation and axial movement of the wing operating member so as to be maintained in the fourth position, and the needle operating member is unlocked from the tube body; and
in the needle driving phase, the needle operating member is driven to move axially from the first position to a second position, so as to drive suturing needles to extend out of the tube body of the tissue suturing device to a puncture position.

19. The operating method of a tissue suturing device according to claim 18, wherein a tactile feedback is provided when the wing operating member is locked to and unlocked from the tube body.

20. The operating method of a tissue suturing device according to claim 18, wherein after the wing operating member is reversely rotated to unlock the wing operating member from the tube body, the wing operating member is either:
manually driven by an operator to move reversely to the third position, or
automatically moved reversely to the third position under the action of energy released by an elastic member,
so as to fold the stabilizing wings.

21.-22. (canceled)
